(19) United States
(12) Patent Application Publication
(10) Pub. No.: US 2011/0014619 A1
(43) Pub. Date: Jan. 20, 2011

(54) REACTION TREATMENT APPARATUS AND REACTION TREATMENT METHOD

(75) Inventor: Yoshimasa Araki, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/826,851

(22) Filed: Jun. 30, 2010

(30) Foreign Application Priority Data
Jul. 16, 2009 (JP) 2009-167906

(51) Int. Cl. C12M 1/42; C12M 1/00; C12Q 1/68

(52) U.S. Cl. 435/6; 435/285.1; 435/285.2

(57) ABSTRACT

Provided is a reaction treatment apparatus in which, in a case of mixing a plurality of solutions in a microchip used in a biochemical reaction system, an electric field generation area for changing solute concentration distribution in the solutions is provided in a solution upstream fluid path. Diffusion between the solutions is accelerated by bringing an area with a high solute concentration into contact with another solution. This may shorten a time required for mixing the solutions.

### BACKGROUND OF THE INVENTION

1. Field of the Invention

The present invention relates to a reaction treatment apparatus and a reaction treatment method. More specifically, the present invention relates to a biochemical reaction treatment apparatus using a microchip having micro structures such as a micro fluid path called a micro channel and a port in a substrate.

2. Description of the Related Art

In recent years, along with advancement of a three-dimensional processing technology, systems have drawn attention, in which liquid elements such as a micro fluid path, a pump, and a valve, and a sensor are integrated on a substrate made of glass, silicon, or the like, and chemical analysis is performed on the substrate. Those systems are known as micro-scale total analysis systems (μTAS). It has been proposed that micro structures such as a micro channel constituting a fluid path in a predetermined shape and a port may be provided in a substrate, and various operations such as chemical reaction, synthesis, purification, extraction, generation, and analysis of a material in the micro structure may be performed, and a part of the operations has been put into practical use. A construction having micro structures such as a micro channel and a port in a substrate, produced for the above-mentioned purpose, is collectively called as a “micro chip”.

Microchips may be used for a wide range of applications such as gene analysis, clinical diagnosis, drug screening, and environmental monitoring. The microchips have the following advantages: (1) amounts of a sample and a reagent to be used are remarkably small; (2) an analysis time is short; (3) the microchips may be carried and perform analysis on the spot; and (4) the microchips are disposable.

In those microchips, a reagent solution, a sample solution, and the like are mixed in a chip, and thereafter, the mixed solution is introduced into a reaction treatment area such as a chamber, followed by heating or cooling, and is allowed to effect a biochemical reaction. Then, the reaction state is detected, and thus, the results of gene analysis, clinical diagnosis, drug screening, environmental monitoring, and the like are detected.

In a conventional microchip, when solutions are mixed in the microchip, two liquids containing reagents are brought into contact with each other to diffuse solutes, and thus, the two liquids are mixed. However, mixing by diffusion of solutes has a problem that a time required for sufficient mixing is long.

As a method of solving the above-mentioned problem, there is a technology of mixing solutions while feeding the solutions into a reaction area. U.S. Pat. No. 5,842,787 discloses a configuration in which two fluid paths for allowing two different liquids to flow are provided on an upstream side, a merged fluid path communicating with the two fluid paths is provided on a downstream side of the two fluid paths, and a reaction area is provided ahead of the merged fluid path. Further, U.S. Pat. No. 5,842,787 discloses a configuration in which, in order to enhance a mixing efficiency of the two liquids at a time of merging, an aspect ratio (width divided by depth) of the fluid path is set at 1 or less so that a contact area of laminar flows generated at a time of merging increases.

However, when the diffusion of solutes is utilized, it requires a time for the solutes present away from the contact surface of the laminar flows to reach another liquid. Therefore, in a case where an attempt is made so as to mix sufficient amounts of solutes efficiently, a long fluid path is inevitably required.

In contrast, Japanese Patent Application Laid-open No. 2007-101289 discloses a configuration in which a micro fluid path in a Y-shape in which liquid flows in from two flow inlets is provided, an AC voltage is applied between electrodes, using a micro fluid device having an electrode whose edge portion is positioned close to the fluid path and a counter electrode positioned away from the fluid path, and thus, a sample and a carrier fluid are allowed to circle in the fluid path to mix two solutions uniformly.

According to a conventional example for mixing solutions in a micro fluid path, two solutions are mixed uniformly, and as a result, the concentration of a solute in one solution is diluted with the other solution. Therefore, in terms of a reaction probability, a sufficient effect may not be obtained due to two contracting influences, that is, the enhancement of a contact probability by uniformalization and the decrease in a contact probability by dilution. Therefore, in some cases, it is necessary to prepare high-concentration solutions considering dilution, and the step of concentration is previously required.

In order to achieve a rapid reaction treatment while achieving miniaturization of a microchip and the further miniaturization of a biochemical reaction apparatus using the microchip, there is a demand for a configuration in which a reaction unit where target materials are present at densities higher than those in the conventional example is formed locally and a plurality of fluids are introduced into a reaction treatment area.

### SUMMARY OF THE INVENTION

The present invention has been made in view of the above-mentioned problems, and therefore has an object to provide a reaction treatment apparatus capable of performing a reaction treatment more efficiently compared with the conventional example.

That is, according to the present invention, there is provided a reaction treatment apparatus for merging a plurality of kinds of fluids containing materials to be used in a reaction treatment and introducing the merged fluids into a downstream reaction treatment area, the reaction treatment apparatus including: a plurality of upstream fluid paths for allowing each of the plurality of kinds of fluids to flow therethrough; a merged fluid path for communicating with the plurality of upstream fluid paths and introducing the merged fluids into the downstream reaction treatment area; and a section for densely gathering the materials in at least one fluid flowing through the merged fluid path on a side in contact with another fluid.

A first aspect of the section for densely gathering the materials is a section for generating concentration distribution of the materials to be used in a reaction treatment in at least one of the plurality of upstream fluid paths with respect to a direction perpendicular to a direction in which the fluids flow.

Further, a second aspect of the section for densely gathering the materials is a section for generating concentration distribution of the materials with respect to a direction perpendicular to a direction in which the fluids flow, placed in the merged fluid path.

Further, according to the present invention, there is provided a reaction treatment method of merging a plurality of kinds of fluids containing materials to be used in a reaction treatment and introducing the merged fluids into a downstream reaction treatment area, the reaction treatment method including densely gathering the materials in at least one fluid on a side in contact with another fluid when merging the plurality of kinds of fluids by allowing the plurality of kinds of fluids to flow from an upstream side using a plurality of upstream paths.

According to the present invention, by densely gathering the materials on a side in contact with another fluid, the area with a high-concentration of a target material may be brought into contact with another solution, and the diffusion between the solutions may be accelerated locally. Consequently, a reaction site in which the target materials are present in a high density may be formed locally, which enhances a reaction efficiency. Further, a solution on a non-dense side formed by the densely gathering section may be prevented from being introduced into a reaction area. This may selectively introduce a mixed solution concentrated at a high concentration into a reaction treatment area.

Further features of the present invention will become apparent from the following description of exemplary embodiments with reference to the attached drawings.

### BRIEF DESCRIPTION OF THE DRAWINGS

FIG. 1 is a perspective view of a biochemical reaction system of Embodiment 1 to which the present invention may be applied.

FIG. 2A is a plan view of a microchip of Embodiment 1 to which the present invention may be applied.

FIG. 2B is a cross-sectional view taken along the line 2B-2B of FIG. 2A.

FIG. 3 is an enlarged view of the vicinity of a merged portion between a first upstream fluid path 9 and a second upstream fluid path 10 of FIG. 2A.

FIG. 4 is an enlarged view of the vicinity of a merged portion between a first upstream fluid path 9 and a second upstream fluid path 10 of Embodiment 2 to which the present invention may be applied.

FIG. 5A is a plan view of a microchip of Embodiment 3 to which the present invention may be applied.

FIG. 5B is an enlarged view of the vicinity of a merged portion between a first upstream fluid path 9 and a second upstream fluid path 10 of FIG. 5A.

FIG. 5C is a view illustrating that a branched fluid path 42 is provided at the upstream fluid path 10 of FIG. 5B.

FIG. 6 is a plan view of a microchip of Embodiment 4 to which the present invention may be applied.

FIG. 7 is a plan view of a microchip of Embodiment 5 to which the present invention may be applied.

### DESCRIPTION OF THE EMBODIMENTS

Preferred embodiments of the present invention will now be described in detail in accordance with the accompanying drawings.

According to the present invention, a reaction treatment refers to a treatment of effecting a reaction irrespective of whether the reaction is a physical, chemical, or biochemical reaction. Examples of combination of materials to be used in the reaction treatment include a solid carrier (beads) and a specimen (nucleic acid, protein, etc.) in a physical reaction treatment (adsorption) and a specimen and a labeled substance in a chemical reaction treatment (binding reaction). Further, in a biochemical reaction treatment, examples of a combination of materials to be used in the reaction treatment include nucleic acid to be detected in polymerase chain reaction (PCR), a nucleic acid primer for PCR, a base for PCR amplification, and polymerase enzyme.

In particular, there are reagents or the like required to be stored individually in a cooled state such as enzyme or the like that is a material used in the biochemical reaction treatment (and biochemical reaction treatment apparatus) and that decreases in activity with the passage of time. The mixing method of the present invention exhibits efficient effects to those reagents.

According to the present invention, examples of a fluid to be used include those in a liquid form, a semi-solid form, and a sol-gel form. Liquid is mainly used. A fluid having a material to be used in a reaction treatment is generally a solution in which the material is dissolved. However, the fluid also includes a state in which the material is suspended.

Further, according to the present invention, in a case of using a reaction apparatus normally, a fluid path placed on an upstream side of a direction in which liquid flows is defined as an upstream fluid path and a downstream side is defined as a reaction treatment area. However, the liquid does not necessarily flow in the same direction at all times.

The present invention has a section for densely gathering materials to be used in a reaction treatment on a side in contact with another fluid in at least one fluid flowing through a merged fluid path. A section for densely gathering materials locally, instead of uniformalizing the materials by stirring such as circling, is used. This forms an area (reaction unit) where a plurality of kinds of materials are present at high concentrations in the merged fluid path and introduces a fluid having the reaction unit into a reaction treatment area, thereby realizing an efficient reaction treatment. More specifically, the effect equivalent to that of a system of mixing two kinds of solutions, at least one of which is set at a high concentration, uniformly is obtained, and hence, time and labor for adjusting a solution previously concentrated at a high concentration are eliminated.

As the densely gathering section, there is a section for generating concentration distribution of the material to be subjected to the reaction treatment in a direction perpendicular to the direction in which the liquid flows. More specifically, an electric field generator can be suitably used. Depending upon the material, concentration distribution may be generated using a magnetic field generator and an ultrasonic generator, and a material to be used for the reaction treatment may be densely gathered on a side in contact with another fluid.

As the electric field generator, there is a configuration of providing an electric field generation area in at least one upstream fluid path. This enables an area in which at least two kinds of materials in different fluids are present in a high solute concentration to be formed in a contact area between the fluids or in the vicinity thereof. Consequently, a reaction unit at a high concentration may be introduced into a reaction area on a downstream side.

Further, by providing the electric field generation area at the merged fluid path as the electric field generator, an area (reaction unit) at a high solute concentration may be formed in the contact area between the fluids or in the vicinity thereof, or in one solution. Consequently, the reaction unit of a high concentration may be introduced into a reaction area on a downstream side.

By providing an electric field generation area of changing a solute concentration distribution in a solution in at least one solution upstream fluid path and providing an electric field generation area in the vicinity of the merged fluid path in the case of mixing a plurality of kinds of different solutions, a ratio at which the densely gathered solutions at a high concentration diffuse in the upstream fluid path may be reduced. This enables a solution at a high concentration to be sent so that the solution comes into contact with another fluid without fail, and mixing in concentration (concentrated mixing) between solutions may be accelerated. Further, by providing an electric field generation area in the vicinity of the merged fluid path, a ratio at which the densely gathered solutions at a high concentration come into contact with a wall surface of an upstream fluid path may be reduced. Consequently, an amount of a solute that adheres to the upstream fluid path before merging may also be reduced.

By providing a branched fluid path in which a part of a solution is branched in an upstream fluid path in which the electric field generator is provided, a solute with the concentration thereof enhanced by the electric field generation area may be prevented from moving to a low-concentration portion. As a result, a solution at a high concentration may be sent to the merged fluid path without fail, and a high-concentration area may be realized.

Further, by providing a branched fluid path in which a part of a solution is branched in the merged fluid path, a solute with the concentration thereof enhanced by the electric field generation area on a side in contact with another fluid may be prevented from moving to a low-concentration portion on the opposite side. As a result, a solution at a high concentration may be sent to the reaction treatment area without fail, and an efficient reaction treatment may be realized.

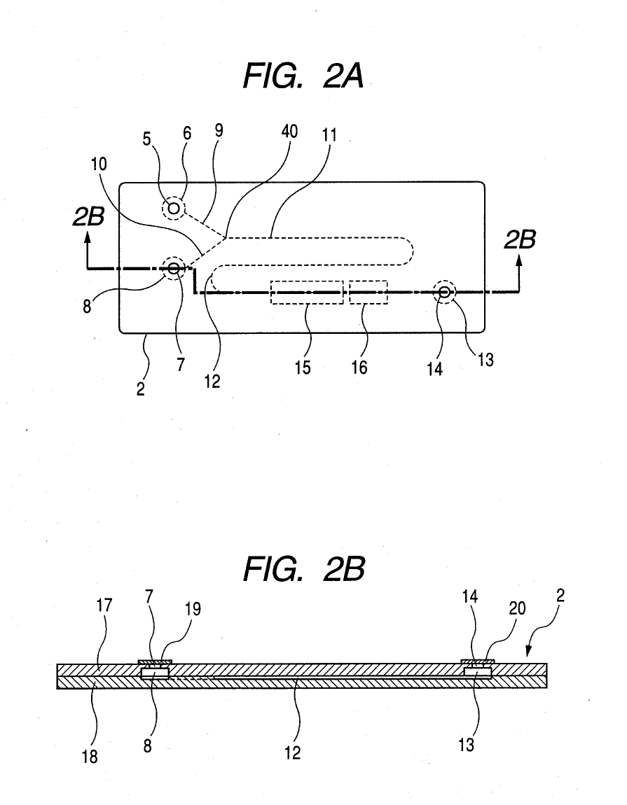

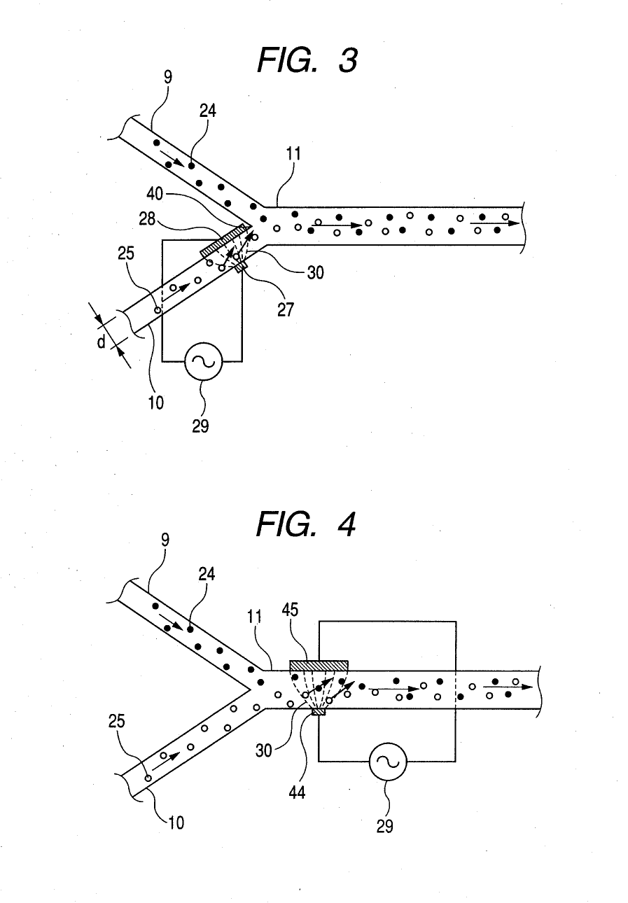

The purpose of placing the electric field generation area is to densely gather materials in fluids on a side where the merged fluids are brought into contact with each other, as described above, and in order to obtain this effect sufficiently, the electric field generation area can be more suitably placed in the vicinity of a merged site 40 (illustrated in FIGS. 2A and 3) at which the upstream fluid path and the merged fluid path are connected. Although a suitable placement range changes depending upon the shape of a fluid path, a flow speed, etc., in a case where a distance between a wall surface on a side where fluids that come into contact with each other at a time of merging flow and the farthest wall surface opposed to the above-mentioned wall surface is d as illustrated in FIG. 3, the electric field generation area may suitably be placed at a distance within 5 times of d from the merged site 40 at which the fluids come into contact with each other for the first time. For example, in the case where d is 100 μm in the configuration illustrated in FIG. 3, an electrode 28 may be placed so that the distance between the merged site 40 and an end of the electrode 28 is within 500 μm.

By changing the solute concentration distribution in a solution by dielectrophoresis, an AC may be used in an electrode generating an electric field and corrosion of an electrode and generation of bubbles at the electrode may be prevented.

The electrode used for dielectrophoresis may be any electrode that may generate a non-uniform electric field. In general, the electrode may be configured by opposing two electrodes having different sizes and shapes to each other.

A plurality of kinds of liquids to be merged in the present invention may be set appropriately depending upon the respective use purposes. For example, even in the case of mixing at least two kinds of liquids having the same composition and different composition concentrations, or in the case of mixing at least two liquids having the same composition concentration and different synthesis periods of enzyme or the like, etc., the present invention may be used. In the reaction treatment such as a biochemical reaction treatment, the configuration of mixing liquids having different storage environments before merging and different processes of treatment may be suitably used. For example, a configuration of merging a material to be treated of a sample containing DNA extracted from a human or an animal with a reagent having enzyme or the like for performing a PCR reaction treatment with respect to the sample may be suitably used.

Further, according to the present invention, one end of the upstream fluid path can be connected to a fluid path for performing a plurality of treatments. For example, in an further upstream portion of the upstream fluid path, a step of extracting nucleic acid from a blood sample, a step of separating nucleic acid, or the like is performed, a sample to be treated is placed at one end of the upstream fluid path, and a reagent such as enzyme for performing PCR reaction treatment is placed at the other end, and thus, the sample and the reagent may be mixed in the merged fluid path of the present invention.

### Embodiment 1

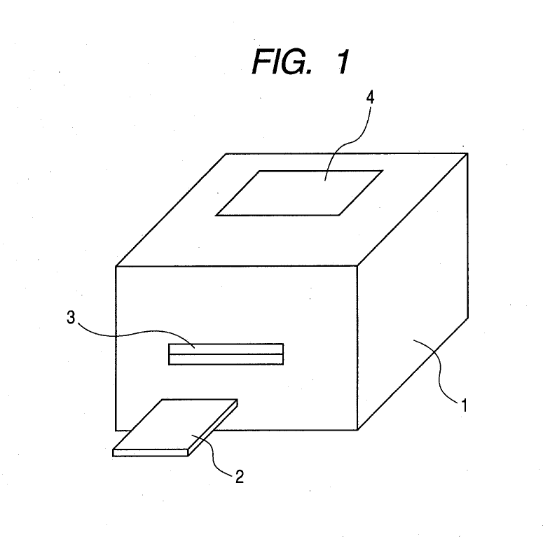

Hereinafter, Embodiment 1 to which the present invention may be applied is described with reference to FIGS. 1, 2A and 2B, and 3.

FIG. 1 is a perspective view of a biochemical reaction system. The biochemical reaction system 1 has a set portion 3 through which a microchip 2 is inserted or discharged. On an upper surface of the biochemical reaction system 1, a display portion 4 displaying an operation state, an inspection result, etc., is provided.

FIG. 2A is a plan view of the microchip. The microchip 2 has a first introduction port 5 and a second introduction port 7, and the first introduction port 5 communicates with a first chamber 6. The first chamber 6 communicates with a first upstream fluid path 9. The first upstream fluid path 9 communicates with a merged fluid path 11. The merged fluid path 11 communicates with a main fluid path 12. The main fluid path 12 communicates with a first waste liquid chamber 13. The first waste liquid chamber 13 communicates with a first opening 14. The second introduction port 7 communicates with a second chamber 8. The second chamber 8 communicates with a second upstream fluid path 10. The second upstream fluid path 10 communicates with the merged fluid path 11. The main fluid path 12 is provided with a thermal treatment area 15 performing a thermal treatment of heating/cooling for effecting a biochemical reaction of a solution in the main fluid path 12. Further, a detection area 16 for detecting a reaction result after the biochemical reaction by heating/cooling is provided on a downstream side of the thermal treatment area 15. Sheets (not shown) are attached to the first introduction port 5, the second introduction port 7, and the first opening 14, respectively. By attaching the sheet, foreign matter is prevented from entering the microchip 2 through the first introduction port 5, the second introduction port 7, and the first opening 14. As described in FIG. 1, when the microchip 2 is inserted in the set portion 3 of the biochemical reaction system 1, openings are formed in the sheets illustrated in FIG. 2A by a perforating member provided in the biochemical reaction system 1. Then, a reagent is introduced into the first introduction port 5 from the opening of the sheet, and a specimen solution is introduced into the second introduction port 7. After the reagent and the specimen solution are introduced, air is sent by a pump via a connection portion connected to the opening of the sheet in an air-tight manner, and thus, the reagent and specimen solution in the first chamber 6 and the second chamber 8 are sent to the upstream fluid paths. An opening is also formed in the sheet attached to the first opening 14 by the perforating member, and thus the first opening 14 may communicate with the atmosphere. Solution sending may be aided by performing suction by the pump via a connection portion in contact with the opening of the sheet attached to the first opening 14 in an air-tight manner.

FIG. 2B is a cross-sectional view taken along the 2B-2B line of the microchip 2 of FIG. 2A. The microchip 2 is configured by thermal bonding of a cover plate 17 and a base plate 18 in an air-tight manner. The cover plate 17 and the base plate 18 may be produced by injection molding using a resin, or a fluid path may be formed in glass by etching. Although not illustrated in FIG. 2A, a first sheet 19 is attached to the second introduction port 7, and a second sheet 20 is attached to the first opening 14.

FIG. 3 is an enlarged view of the vicinity of the merged portion between the first upstream fluid path 9 and the second upstream fluid path 10 of FIG. 2A. On the second upstream fluid path 10, a first electrode 27 and a second electrode 28 are provided so as to be opposed to each other. The first electrode 27 and the second electrode 28 are electrically connected to an AC power source 29. The first electrode 27 and the second electrode 28 are provided so as to be opposed to each other in the vicinity of the merged fluid path 11 of the second upstream fluid path 10. A portion of the first electrode 27 in which the first electrode 27 is brought into contact with the second upstream fluid path 10 is formed smaller than a portion of the second electrode 28 in which the second electrode 28 is brought into contact with the second upstream fluid path 10. Since the portion in which the first electrode 27 is brought into contact with the second upstream fluid path 10 is different in size from the portion in which the second electrode 28 is brought into contact with the second upstream fluid path 10, a non-uniform electric field is generated between the first electrode 27 and the second electrode 28. More specifically, there is a coarse and fine part in a line of electric force 30 between the first electrode 27 and the second electrode 28. A reagent containing a base or a primer 24 (solid black circles all indicate a base or a primer) that is a material used in a reaction treatment is sent from the first chamber 6 illustrated in FIG. 2A to the first upstream fluid path 9. A solution containing a specimen 25 (solid white circles all indicate a specimen) that is a material used in another reaction treatment is sent from the second chamber 8 illustrated in FIG. 2A to the second upstream fluid path 10. The specimen in the vicinity of the first electrode 27 and the second electrode 28 is deflected in a direction indicated by arrows of FIG. 3 due to the influence (dielectrophoresis) of an electric field generated between the first electrode 27 and the second electrode 28. The deflected specimen is introduced into the merged fluid path 11 in a concentrated state in the specimen solution.

The dielectrophoresis is one electrodynamics phenomenon, and according to the dielectrophoresis, a force acts on particles due to the interaction between a dipole moment in particles induced by a non-uniform electric field applied from outside and an external electric field. Unlike the electrophoresis, the dielectrophoresis does not depend upon charges owned by the particles but depends upon an applied frequency, an applied voltage, the conductivity and permittivity of a solvent and particles, and the size of fine particles. The direction of the force acting on the particles changes depending upon the frequency. Depending upon the frequency, there are a case where positive dielectrophoresis acts and fine particles move in a direction of the highest electric field intensity and a case where negative dielectrophoresis acts and fine particles receive a repulsive force from an area of a high electric field intensity to move in a direction of a low electric field intensity. In the case of positive dielectrophoresis, fine particles may adsorb to an electrode for generating an electric field. Therefore, according to the present invention, fine particles are deflected by negative dielectrophoresis. (Reference literature: BUNSEKI KAGAKU Vol. 54, No. 12, pp. 1189-1195).

As a result that the specimen is concentrated in the specimen solution, the specimen solution comes into contact with the reagent solution in a state in which the concentration of the specimen at an interface with the reagent solution is high.

Diffusion is phenomenon in which particles move in a direction in which a solute is diluted in a solution. The amount of a solute diffusing in a unit time through a unit area is proportional to the gradient of a concentration. Therefore, in a case where concentration difference of a solute between solutions is large, the amount of a diffusing solute increases, compared with a case where concentration difference is small. Then, as the distance from the diffusing solute to an area to which the solute should diffuse is shorter, the solute diffuses in a shorter period of time. Therefore, compared with a case where the concentration of a specimen is not increased, a reagent containing a base or a primer is brought into contact with a specimen solution in a state in which the concentration of the specimen at an interface with the reagent solution is high, and thus, the local diffusion is accelerated and mixing may be performed in a shorter period of time.

The specimen in the vicinity of the first electrode 27 and the second electrode 28 is deflected in a direction indicated by arrows of FIG. 3 due to the influence (dielectrophoresis) of an electric field generated between the first electrode 27 and the second electrode 28. When the first electrode 27 and the second electrode 28 are placed in the vicinity of the merged fluid path 11, the concentrated specimen is introduced into the merged fluid path 11 before diffusing in the upstream fluid path 10. The concentrated specimen may be mixed in a high concentration state without diffusing, which enhances the mixing efficiency in a portion which the fluid is in contact. Further, by providing the first electrode 27 and the second electrode 28 in the vicinity of the merged fluid path 11, the area in which the solution containing a high-concentration specimen comes into contact with the upstream fluid path wall, surface may be reduced. Consequently, the amount of the solute adhering to the upstream fluid path 10 may be reduced. As illustrated in FIG. 3, the first electrode 27 and the second electrode 28 are suitably be provided in the vicinity of the merged fluid path 11, as described above.

### Embodiment 2

Hereinafter, Embodiment 2 to which the present invention may be applied is described with reference to FIG. 4.

FIG. 4 is an enlarged view of the vicinity of a merged portion of upstream fluid paths. In Embodiment 2, positions of a first electrode 44 and a second electrode 45 are changed with respect to Embodiment 1. The remaining configuration is the same as that of Embodiment 1. The merged fluid path 11 is provided with the first electrode 44 and the second electrode 45 opposed to each other. The first electrode 44 and the second electrode 45 are electrically connected to the AC power source 29. A portion of the first electrode 44 in which the first electrode 44 is brought into contact with the merged fluid path 11 is formed smaller than a portion of the second electrode 45 in which the second electrode 45 is brought into contact with the merged fluid path 11. Since the portion in which the first electrode 44 is brought into contact with the merged fluid path 11 is different in size from the portion in which the second electrode 45 is brought into contact with the merged fluid path 11, a non-uniform electric field is generated between the first electrode 44 and the second electrode 45. More specifically, there is a coarse and fine part in the line of electric force 30 between the first electrode 44 and the second electrode 45. A reagent containing the base or the primer 24 (solid black circles all indicate a base or a primer) is sent to the first upstream fluid path 9. A solution containing the specimen 25 (solid white circles all indicate a specimen) is sent to the second upstream fluid path 10. The specimen in the vicinity of the first electrode 44 is deflected in a direction indicated by arrows of FIG. 4 due to the influence (dielectrophoresis) of an electric field generated between the first electrode 44 and the second electrode 45. The deflected specimen moves to a side of the reagent containing a base or a primer and is concentrated. Consequently, diffusion is accelerated between the portion of a high-concentration specimen and the reagent containing a base or a primer, and mixing may be performed in a short period of time. At a position away from the first electrode 44, a change of coarseness and fineness of the line of electric force is poor. Therefore, the base or the primer 24 are unlikely to be deflected due to the influence (dielectrophoresis) of an electric field.

### Embodiment 3

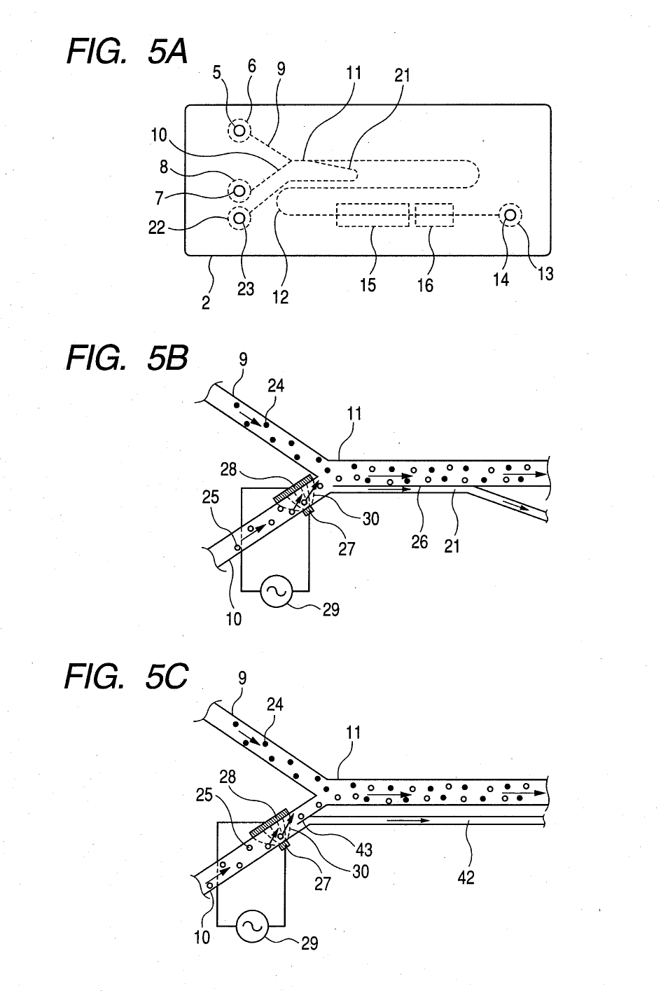

Hereinafter, Embodiment 3 to which the present invention may be applied is described with reference to FIGS. 5A, 5B, and 5C.

FIG. 5A is a plan view of a microchip 2. In Embodiment 3, a first branched fluid path 21, a second waste liquid chamber 22, and a second opening 23 are added to Embodiment 1. The remaining configuration is the same as that of Embodiment 1. The merged fluid path 11 communicates with the first branched fluid path 21. The first branched fluid path 21 communicates with the second waste liquid chamber 22. The second waste liquid chamber 22 communicates with the second opening 23. A sheet (not shown) is attached to the second opening 23 so as to prevent foreign matter from entering the microchip 2 through the second opening 23. As described with reference to FIGS. 1 and 2A in Embodiment 1, when the microchip 2 is inserted in the set portion 3 of the biochemical reaction system 1, openings are formed in the sheets by the perforating member provided in the biochemical reaction system 1. Solution sending may be aided by performing suction by the pump via a connection portion in contact with the opening of the sheet attached to the second opening 23 in an air-tight manner.

FIG. 5B is an enlarged view of the vicinity of the merged portion of the first upstream fluid path 9 and the second upstream fluid path 10 of FIG. 5A. The second upstream fluid path 10 is provided with the first electrode 27 and the second electrode 28 opposed to each other. The first electrode 27 and the second electrode 28 are electrically connected to the AC power source 29. The first electrode 27 and the second electrode 28 are provided in the vicinity of the merged fluid path 11 of the second upstream fluid path 10 so as to be opposed to each other. A portion of the first electrode 27 in which the first electrode 27 is brought into contact with the second upstream fluid path 10 is formed smaller than a portion of the second electrode 28 in which the second electrode 28 is brought into contact with the second upstream fluid path 10. Since the portion in which the first electrode 27 is brought into contact with the second upstream fluid path 10 is different in size from the portion in which the second electrode 28 is brought into contact with the second upstream fluid path 10, a non-uniform electric field is generated between the first electrode 27 and the second electrode 28. More specifically, there is a coarse and fine part in the line of electric force 30 between the first electrode 27 and the second electrode 28. The merged fluid path 11 is provided with a fluid branching wall 26, and thus, the first branched fluid path 21 branched by the fluid branching wall 26 is provided. A reagent containing the base or the primer 24 (solid black circles all indicate a base or a primer) is sent from the first chamber 6 illustrated in FIG. 5A to the first upstream fluid path 9. A solution containing the specimen 25 (solid white circles all indicate a specimen) is sent from the second chamber 8 illustrated in FIG. 5A to the second upstream fluid path 10. The specimen in the vicinity of the first electrode 27 and the second electrode 28 is deflected in a direction indicated by arrows of FIG. 5B due to the influence (dielectrophoresis) of an electric field generated between the first electrode 27 and the second electrode 28. The deflected specimen is introduced into the merged fluid path 11 in a concentrated state in the specimen solution. A solvent portion of the solution containing the specimen is introduced into the first branched fluid path 21.

The specimen is concentrated in the specimen solution, and consequently, comes into contact with the reagent solution in a state in which the concentration of the specimen at the interface with the reagent solution is high.

Compared with the case where the concentration of a specimen is not increased, a reagent containing a base or a primer is brought into contact with a specimen solution in a state in which the concentration of the specimen at an interface with the reagent solution is high, and thus, the diffusion is accelerated and mixing may be performed in a short period of time. By introducing a solvent portion of the solution containing the specimen into the first branched fluid path 21, the specimen in the merged fluid path 11 may be prevented from diffusing to the solvent portion introduced into the first branched fluid path 21 in the merged fluid path 11. Consequently, the specimen solution and the reagent containing a base or a primer may be brought into contact with each other without fail in a state in which the concentration of the specimen at the interface with the reagent solution is high, and hence, the diffusion is accelerated and mixing may be performed in a short period of time.

The specimen in the vicinity of the first electrode 27 and the second electrode 28 is deflected in a direction indicated by arrows of FIG. 5B due to the influence (dielectrophoresis) of an electric field generated between the first electrode 27 and the second electrode 28. When the first electrode 27 and the second electrode 28 are placed in the vicinity of the merged fluid path 11, the concentrated specimen 25 is introduced into the merged fluid path 11 without diffusing. Consequently, the specimen 25 entering the first branched fluid path 21 after reaching the merged fluid path 11 is reduced. The solution in the first branched fluid path 21 is not used for mixing, and hence, it is desired that the amount of the specimen to be introduced into the first branched fluid path 21 be small. When the concentrated specimen diffuses to decrease the concentration, the mixing efficiency decreases, which is not preferred. Further, when the first electrode 27 and the second electrode 28 are provided in the vicinity of the merged fluid path 11, the area in which the solution of a high-concentration specimen comes into contact with the upstream fluid path wall surface may be reduced. Consequently, the amount of a solute adhering to the upstream fluid path 11 may be reduced.

As illustrated in FIG. 5B, the first electrode 27 and the second electrode 28 can be provided in the vicinity of the merged fluid path 11.

FIG. 5C illustrates a state in which the first electrode 27 and the second electrode 28 are moved, a fluid branching wall 43 is provided in the upstream fluid path 10, and a first branched fluid path 42 branched from the upstream fluid path 10 by the fluid branching wall 43 is provided in FIG. 5B. The specimen in the vicinity of the first electrode 27 and the second electrode 28 is deflected in a direction indicated by arrows of FIG. 5C due to the influence (dielectrophoresis) of an electric field generated between the first electrode 27 and the second electrode 28. The deflected specimen is introduced into the merged fluid path 11 through the upstream fluid path 10 in a state in which the deflected specimen is concentrated in the specimen solution. A solvent portion of the solution containing the specimen is introduced into the first branched fluid path 42. By introducing the solvent portion of the solution containing the specimen into the first branched fluid path 42, the concentrated specimen may be prevented from diffusing to the solvent portion in the first branched fluid path 42. Consequently, the specimen solution and the reagent containing a base or a primer may be brought into contact with each other without fail in a state in which the concentration of the specimen at the interface with the reagent solution is high, and a mixed state at a locally high concentration is formed.

### Embodiment 4

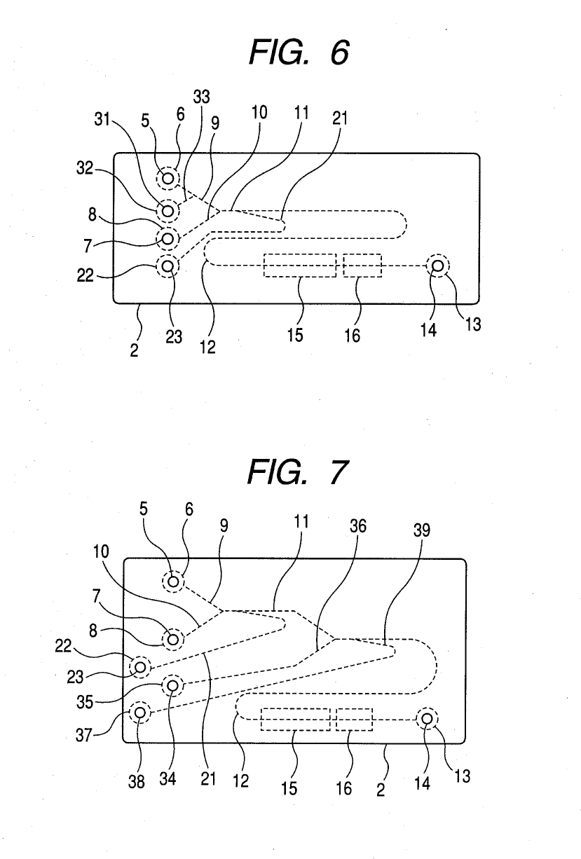

Hereinafter, Embodiment 4 to which the present invention may be applied is described with reference to FIG. 6.

FIG. 6 is a plan view of a microchip of Embodiment 4. Embodiment 4 is different from Embodiment 3 in that a third introduction port 31, a third chamber 32, and a third upstream fluid path 33 are provided to the microchip 2 of Embodiment 3. The remaining configuration is the same as that of Embodiment 3, and hence, the descriptions thereof are omitted.

In FIG. 6, the third introduction port 31 communicates with the third chamber 32. The third chamber 32 communicates with the third upstream fluid path 33. The third upstream fluid path 33 communicates with the first upstream fluid path 9. With such a configuration, two kinds of reagents may be supplied in addition to the specimen. Further, the addition of the introduction port, the chamber, and the upstream fluid path may increase the number of reagents which may be supplied.

### Embodiment 5

Hereinafter, Embodiment 5 to which the present invention may be applied is described with reference to FIG. 7.

FIG. 7 is a plan view of a microchip of Embodiment 5. Embodiment 5 is different from Embodiment 3 in that a fourth introduction port 34, a fourth chamber 35, a fourth upstream fluid path 36, a second branched fluid path 39, a third waste liquid chamber 37, and a third opening 38 are provided to the microchip 2 of Embodiment 3. The remaining configuration is the same as that of Embodiment 3, and hence, the descriptions thereof are omitted.

In FIG. 7, the fourth introduction port 34 communicates with the fourth chamber 35. The fourth chamber 35 communicates with the fourth upstream fluid path 36. The fourth upstream fluid path 36 communicates with the merged fluid path 11. The second branched fluid path 39 communicates with the merged fluid path 11. The second branched fluid path 39 communicates with the third waste liquid chamber 37. The second waste liquid chamber 37 communicates with the third opening 38. The fourth upstream fluid path 36 is provided with electrodes in the same way as in the second upstream fluid path 10 of Embodiment 2. With such a configuration, a solution containing a solute capable of being concentrated by dielectrophoresis may be introduced into the fourth introduction port 34, and the solute may be concentrated in the fourth upstream fluid path 36 and may be introduced into the merged fluid path 11. Then, the mixing of the solution introduced into the merged fluid path 11 from the first upstream fluid path 9 and the second upstream fluid path 10 with the solution introduced into the merged fluid path 11 from the fourth upstream fluid path 36 may be accelerated. A part of the solvent of the solution introduced into the fourth introduction port 34 is distributed to the second branched fluid path 39.

Further, the addition of the introduction port, the chamber, the upstream fluid path, the branched fluid path, the waste liquid chamber, and the opening may increase the kinds of reagents capable of being supplied.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-167906, filed Jul. 16, 2009, which is hereby incorporated by reference herein in its entirety.

What is claimed is:

1. A reaction treatment apparatus for merging a plurality of kinds of fluids containing materials to be used in a reaction treatment and introducing the merged fluids into a downstream reaction treatment area,
the reaction treatment apparatus comprising:
a plurality of upstream fluid paths for allowing each of the plurality of kinds of fluids to flow therethrough;
a merged fluid path for communicating with the plurality of upstream fluid paths and introducing the merged fluids into the downstream reaction treatment area; and
a section for densely gathering the materials in at least one fluid flowing through the merged fluid path on a side in contact with another fluid.

2. A reaction treatment apparatus according to claim 1, wherein the section for densely gathering the materials comprises a section for generating concentration distribution of the materials in at least one of the plurality of upstream fluid paths with respect to a direction perpendicular to a direction in which the fluids flow.

3. A reaction treatment apparatus according to claim 1, wherein the section for densely gathering the materials comprises a section for generating concentration distribution of the materials with respect to a direction perpendicular to a direction in which the fluids flow, placed in the merged fluid path.

4. A reaction treatment apparatus according to claim 2, wherein the section for generating concentration distribution comprises an electric field generator having an electric field generation area.

5. A reaction treatment apparatus according to claim 4, wherein the electric field generation area is provided in a vicinity of a merged site at which the plurality of upstream fluid paths are connected to the merged fluid path.

6. A reaction treatment apparatus according to claim 1, wherein at least one of the plurality of upstream fluid paths is provided with a branched fluid path for distributing a part of the fluids.

7. A reaction treatment apparatus according to claim 1, wherein the merged fluid paths is provided with a branched fluid path for distributing a part of the fluids.

8. A reaction treatment apparatus according to claim 4, wherein the electric field generator comprises a section for inducing dielectrophoresis of the materials to be used in the reaction treatment.

9. A reaction treatment apparatus according to claim 1, wherein the downstream reaction treatment area has a thermal treatment area for performing one of heating and cooling.

10. A reaction treatment apparatus according to claim 1, wherein the downstream reaction treatment area has a treatment area for performing detection of a reaction.

11. A reaction treatment apparatus according to claim 1, wherein the reaction treatment comprises a biochemical reaction treatment.

12. A reaction treatment apparatus according to claim 1, wherein the materials to be used in the reaction treatment comprise at least one of nucleic acid to be detected, a nucleic primer for PCR, a base for PCR amplification, and polymerase enzyme.

13. A reaction treatment method of merging a plurality of kinds of fluids containing materials to be used in a reaction treatment and introducing the merged fluids into a downstream reaction treatment area,
the reaction treatment method comprising densely gathering the materials in at least one fluid on a side in contact with another fluid when merging the plurality of kinds of fluids by allowing the plurality of kinds of fluids to flow from an upstream side using a plurality of upstream fluid paths.
